(19) United States
(12) Patent Application Publication
(10) Pub. No.: US 2009/0149792 A1
(43) Pub. Date: Jun. 11, 2009

(54) COMPOSITION FOR WOUND MANAGEMENT

(75) Inventors: Owen L. Johns, Madeira Beach, FL (US); Gregory Moffat, St. Petersburg, FL (US)

Correspondence Address: DENNIS G. LAPOINTE;LAPOINTE LAW GROUP, PL, PO BOX 1294, TARPON SPRINGS, FL, 34688-1294, US

(73) Assignee: KREETECH INTERNATIONAL CORP., St. Hubert (CA)

(21) Appl. No.: 11/951,509

(22) Filed: Dec. 6, 2007

(51) Int. Cl. A61F 13/02; A61L 15/40

(52) U.S. Cl. 602/48

(57) ABSTRACT

A wound dressing composition and a method of using that composition to enhance the protection and isolation of a minor wound. The composition contains at least one ingredient selected from mature Bamboo stalk pulp or derivatives thereof, an adhesive that adheres to skin selected from the families of synthetic and natural elastomers and a hydrophilic compound.

### FIELD OF THE INVENTION

This invention relates to a dressing for wound protection using an external wound dressing composition containing bamboo pulp from the mature plant stalks or bamboo derivatives that are made by dissolving the bamboo pulp and then regenerating it in a chemical process.

### BACKGROUND OF THE INVENTION

As the understanding of the healing process has progressed, various theories have been advanced regarding the most advantageous ways to protect wounds and to optimize the healing process. For many years, absorptive fabric wound dressings were used because it was generally believed that wounds required air drying or desiccation to promote epithelial resurfacing without infection. Subsequently, however, it was found that moist conditions were preferable for the promotion of healing and absorptive adhesives were developed. Studies have shown that it is preferable to maintain a fluid environment over the wound site in order to promote optimum wound healing. No increase in infection has been associated with wet wound healing. In the case of wounds suspected of infection it is also desirable to shield the patients surrounding skin as well as other persons from contagious microorganisms in the wound fluid.

Providing a dressing device that protects a wound is challenging in that wounds are not uniform. In some cases patients are active. Wounds also have different areas to consider where wound management is concerned. One area is the surrounding intact skin. This area is used to adhere the dressing over the open wound bed. Adhesives that adhere to skin must provide adequate adhesion without harming the skin upon removal. The adhesive formulation must also be non-adherent to the wound bed if it extends over that area. Dressings are commercially available that accomplish this and as such, meet the specific requirement. The second area of concern is the wet wound area. This area initially will be moist during the first stage of wound healing or resurfacing thus challenging the dressing device to absorb some amount of fluid. Many commercially available dressings are somewhat absorptive but they may absorb too little liquid or allow fluid to escape. Possibly contaminated liquid in the latter case may be free to flow out the sides or through the covering of the pad material. Such design inadequacies of common first aid dressings include porous backing materials made of perforated vinyl or woven fabric, non-absorptive films to prevent the pad from sticking to the wound and exposed edges of the pad. This presents an opportunity for transfer of infective organisms in blood such as the HIV virus. Also a new class of staphylococcus known as antibiotic resistant community acquired (MRSA) has emerged. First isolated in native American communities in the Midwestern USA, this new organism seems to be spreading rapidly and requires a much more protective and effective dressing than first aid dressings known in the prior art. Preferably the dressing should still allow social and sports activities of the injured individual.

Other factors must be considered in the development of wound dressings which occlude and provide a fluid environment over the wound site. For example, wound dressings must be easily sterilized and stored. Further, wound dressings should be at least toxicologically unobjectionable and be as biocompatible as possible with the human body and not support bacterial growth. Heavy metals and cytotoxic antiseptics cause tissue damage when occluded. Excessive use of antibiotics appears to be the very cause of antibiotic resistant staphylococcus.

This leads to the need for an organic antibacterial or bacteriostatic material. Further considerations in the development of occluded wound dressings include sufficient gas permeability for ventilation of water vapor from the wound and surrounding skin area; non-adherence to the regenerating tissue of the wound, a high level of absorbability for wound exudate fluid and as previously stated, the dressing must be conducive to promoting granulation tissue or re-epithelization necessary to restore the natural protection of new skin growth.

It is important to strike a balance between all of these competing factors in the development of wound dressings which promote wound healing. Of particular importance is a dressing's effect on the healing process. For example, many wound dressings, if allowed to dry over a wound site will stick to the wound. When the adhering dressing is removed from the wound, damage to the integrity of the new tissue at the wound site may result. Further, if a wound is allowed to dry out due to inadequate coverage by a dressing, the healing process is compromised and new tissue may dehydrate and die. Simply filling the wound with harsh biocides such as iodine or heavy metals may actually cause further injury to the tissues and delay healing if covered. Recently we have seen that over use of antibiotics has caused a mutation of antibiotic resistant organisms and protection must be supplied by new means.

In an attempt to facilitate wound healing, different dressings and wound management regimes have been developed. Wound management devices have utilized many materials including cotton gauze, tapes, film dressings, hydrocolloid absorptive adhesives, gels, foams and saline solutions or other pharmaceutically acceptable materials in an attempt to promote wound healing. Less attention has been given to isolating the wound from the patients infectious surroundings since in the past, most opportunistic organisms could be curtailed with antibiotics.

Numerous approaches, having varying degrees of success, have attempted to overcome deficiencies in the prior art wound dressings. Specifically, various saccharide containing wound dressings have been developed which exhibit moisture absorption capabilities. For example, U.S. Pat. No. 4,929,577 to Cornell, U.S. Pat. No. 4,883,478 to Lerailler et al., U.S. Pat. No. 4,788,237 to Le-Khac, U.S. Pat. No. 4,664,105 to Dautzenberg et al., U.S. Pat. No. 4,556,056 to Fischer et al., U.S. Pat. Nos. 4,307,717 and 4,306,551 to Hymes et al. and U.S. Pat. No. 2,137,169 to Levey disclose various saccharide containing wound dressings. However, in all of these dressings there are substantial manufacturing limitations and there is a concern that they could support bacterial growth in use.

In view of the prior art at the time the present invention was made, it was not obvious to those of ordinary skill in the pertinent art how an economical dressing could simultaneously cover a wound and not allow bacterial growth while avoiding strong bactericides or antibiotics.

### SUMMARY OF THE INVENTION

As mentioned above, this invention relates to a dressing for wound protection using an external wound dressing composition containing bamboo pulp from the mature plant stalks or bamboo derivatives that are made by dissolving the bamboo pulp and then regenerating it in a chemical process. This transforms a natural material into a man made product. The natural bacteria resistance of the bamboo plant may be enhanced through further processing, and surprisingly, in combination with adhesive mixtures providing skin adhesion and suitable moisture absorbents, an outstanding wound covering is produced. Such compositions uniquely protect against bacterial growth and proliferation in first aid applications.

In accordance with the present invention, bamboo pulp and bamboo pulp regenerated into fibers has shown resistance to bacterial growth. Chemical processes can further enhance the bacteriostatic property. Incorporating a natural bacteria resistant plant material as part of a hydrocolloid adhesive mixture inhibits the growth of bacteria in contact with the wound covering.

Some bacteria in the wound fluid solution are absorbed into a hydrocolloid adhesive mixture. Free to migrate and attracted by the bamboo absorbance, (Bamboo absorbs up to 10 times dry weight but does not dissolve or form a gel), bacteria brought in contact with the fiber is then rendered harmless.

Since the bamboo molecules are relatively large and insoluble, they do not migrate into the wound. Even if a bamboo fiber were to be incorporated into human tissue, it is biocompatible and readily absorbed by the body. Thus the present invention accomplishes bacteriastasis without the danger of heavy metals or soluble chemicals entering the body.

The absorptive function of bamboo is substituted for some of the absorbents common in the prior art hydrocolloid dressing formulae. There are other unique physical properties of bamboo fiber such as insolubility, small fiber size, lack of chemical reactivity, attraction to protein and absence of toxicity presenting a collection of properties that provide a surprising extension to the functionality of absorbent adhesive wound dressings.

The invention accordingly will be exemplified without limit in the description hereinafter set forth, and the scope of the invention will be indicated in the claims.

### DETAILED DESCRIPTION OF THE INVENTION

The following examples and descriptions will now more fully illustrate and explain certain and various embodiments of the present invention, percentages therein being by weight, unless otherwise noted. The wound covering adhesives associated with this novel present invention are made according to mixing processes generally known in the art such as sigma blade and Banbury mixers. The mixed adhesive is then spread using a feed screw extruder and slot die. The sheet of absorbent adhesive is finally converted into a dressing by laminating and cutting.

### EXAMPLE 1

An example of a preferred bamboo absorptive adhesive wound dressing formulation made from bamboo pulp is:

Bamboo processed pulp (about 35%); Polyisobutylene (about 45%); Pectin (about 10%); Carboxymethylcellulose—CMC (about 8%); and Polybutene (about 2%).

The amount of bamboo pulp which may be used in the above formulation may vary from about 26% to 44.0% w/w by varying the equivalent amounts of pectin and CMC. The pulp would be powdered or ground to a screen size of 100 to 200 (Tyler), preferably 150 mesh.

### EXAMPLE 2

Another example of a bamboo adhesive wound dressing formulation made from regenerated fibers allowing a thin sheet of adhesive to be made is as follows: Pectin (about 20%); CMC (about 10%); Mineral Oil (about 4%); Bamboo fiber flock (about 26%); and Polyisobutylene (about 40%).

The amount of bamboo derivative which may be used in the above formulation, may range from about 11% to 41% by adjusting the ration of pectin and CMC absorbents. The dry bamboo component should be chopped fibers of about 0.5 to 3.0 mm in length, preferably about 1.5 to 2.0 mm.

When the formulation is placed over a wound site, the dry adhesives adhere to the intact skin surrounding the wound to hold the dressing in place over the wound bed. The wet adhesive (pectin) seals around the edge of the wound while the absorbent bamboo component and super absorbent (CMC) cover the wound site and absorb excess wound fluid.

Generally, an oxygen and vapor-permeable thin film outer layer with an adhesive coating will be laminated over the bamboo derivative hydrocolloid formulation on at least a portion thereof for preventing adhesion to clothing or bed linens. It may also be extended beyond the borders of the hydrocolloid adhesive to act as a second seal against ingress or egress of fluids from the wound. The outer layer, which may be made of any suitable material, such as adhesive coated polyurethane or ethylene vinyl acetate, from 0.5 to 5 mils thick, also serves as a support for the dressing prior to application. In addition, it acts as a moisture barrier to prevent drying of the hydrocolloid dressing during the dressing's shelf life. A protective package for storage may be provided by suitable material such as, metal foil, plastic wrap or other suitable packaging known to those skilled in the art.

The dressing may contain other components, compounds or ingredients for performing specifically desired additional functions, for example, super absorbents, guar gum, alginates, C5 tackifiers, fillers, or other bioactive agents.

The outer oxygen and vapor-permeable layer, also serves as a bacterial barrier, and is preferably transparent. Suitable films, adhesives and their preparations are described, for example, in U.S. Pat. No. 3,645,835, which is incorporated herein by reference. The adhesive coated oxygen- and vapor-permeable layer should preferably have a water vapor transmission rate (WVTR) of at least 250 g/m/24 hrs (40.degree. C, 80% relative humidity (RH)). Especially preferred are such adhesive coated oxygen- and vapor-permeable films with a WVTR of about 400 to 500 g/m.sup.2/24 hrs in which the backing material is a transparent polyurethane film having a thickness of about 0.5 to 2 mils (13 to 51 microns) and the film is coated with a 1 mil (25 microns) layer of pressure-sensitive acrylic ester copolymer adhesive.

The water proof outer-film layer is typically made from synthetic polymers or coated non-woven or woven materials which are capable of being formed into continuous sheets by casting, extrusion or other known film making processes. The film is also preferably conformable to body surfaces. Conformability is somewhat dependent on thickness, thus the thinner the film the more conformable it is. In a preferred embodiment the film thickness is from 0.5 to 5 mils. Films of this type are known in the art and generally are hydrophilic, polymeric materials such as polyether block amides, copolymers of cyclic polyesters, elastomeric polyesters, blends of polyurethane and polyester, chlorinated polyethylene, styrene/butadiene block copolymers, polyvinyl chloride and other commercial polyurethane compositions. Nonwoven sheet materials with pore diameters below twenty (20) microns could be useful in the present invention if such nonwoven materials were coated on one face with a thin bacteria proof layer of polymer. Further, the polymer film must be continuous in that it has no perforations or pores in the film.

The adhesive coating for the film may be selected from any number of commercially available medical grade adhesives known in the art. For example, multipolymer emulsions comprising stable pressure-sensitive aqueous acrylic adhesives having a solids content of 59% and a viscosity of 1,500-Z, 300 CPS are useful in the present invention. The adhesive properties of medical grade adhesives can be adjusted by the addition of a greater amount of cross-linking additives and/or by utilizing different coating weights and/or viscosities of the adhesive materials.

The adhesive coating may contain other components or reagents for performing specific desired functions, for example, tackifiers, fillers, medicaments or other bioactive agents. The adhesive coating should be as thin as possible while functioning to adhere the dressing to the skin. Preferably, it is from 0.5 to 5 mils thick.

Generally, a release sheet, preferably silicone release paper, is releasably secured to protect the skin contact hydrocolloid dressing element prior to application of the dressing to a wound site. During application, the release sheet is removed, the dressing is then applied to the wound. Specifically, the release sheet can be made from numerous commercially available silicone or flurocarbon coated release sheets known in the art. Base polymers and papers, such as polyester, polypropylene, polyethylene, styrene, unbleached and bleached kraft papers which can be clay coated or uncoated are also useful in the present invention. Numerous materials known in the art would be suitable based on factors including the need for transparency, stiffness and release force from a chosen adhesive. Additionally, the release sheet can be made of polyethylene, polypropylene or polyester which is coated with a releasing agent such as silicone or fluorochemicals. Preferred release layers are silicone coated.

The absorbent dressing bamboo component or compound may be made of bamboo pulp, fiber, fiber cut into flock or fabric type presentations in combination with natural and synthetic polymeric absorbents, hydrocolloid/polysaccharide absorbents, gum absorbents, resin absorbents, inorganic absorbents and hydrogel absorbents.

The wound dressings of the present invention are particularly useful in wound management regimes that require frequent dressing changes since they do not stick to the wound itself. The dressings of the present invention facilitate the outward movement of wound exudate while maintaining the moisture and warmth of the wound epithelium and surrounding skin.

It will thus be seen from the foregoing description, that certain changes may be made without departing from the scope of the invention. It is intended that all matters contained in the foregoing description shall be interpreted as illustrative and not in a limiting sense. It is also to be understood that the following claims are intended to cover all of the generic and specific features of the invention herein described, and all statements of the scope of the invention which, as a matter of language, might be said to fall there between. It should therefore be understood that the preceding is merely a detailed description of one or more embodiments of this invention and that numerous changes to the disclosed embodiments can be made in accordance with the disclosure herein without departing from the spirit and scope of the invention. The preceding description, therefore, is not meant to limit the scope of the invention. Rather, the scope of the invention is to be determined only by the appended claims and their equivalents.

What is claimed is:

1. A self adhesive topically applied wound site dressing comprising a hydrocolloid adhesive admixture of:
at least one dry adhesive that adheres to dry skin;
at least one wet adhesive that adheres to wet skin;
at least one moisture absorbing hydrophilic compound; and
a wound dressing composition containing bamboo pulp and/or bamboo pulp regenerated as fibers and/or a derivative of said bamboo pulp and/or bamboo pulp regenerated as fibers,
wherein said admixture is in an amount sufficient to protect the wound site during dehydration or desiccation and from the effects of dehydration or desiccation and to inhibit bacteria growth in the dressing and wound covering.

2. The wound site dressing according to claim 1, wherein the wound dressing composition containing bamboo pulp and/or bamboo pulp regenerated as fibers and/or a derivative of said bamboo pulp and/or bamboo pulp regenerated as fibers is about 11% to about 44% by weight of the adhesive admixture.

3. The wound site dressing according to claim 1, wherein the wound dressing composition containing bamboo pulp and/or bamboo pulp regenerated as fibers and/or a derivative of said bamboo pulp and/or bamboo pulp regenerated as fibers is processed to a fiber size of about 0.5 to 3.0 mm in length prior to adding to said admixture.

4. The wound site dressing according to claim 1, further comprising an oxygen and vapor-permeable thin outer film with an adhesive coating laminated over at least a portion of said admixture.

5. The wound site dressing according to claim 1, wherein said wound dressing composition containing bamboo pulp and/or bamboo pulp regenerated as fibers and/or a derivative of said bamboo pulp and/or bamboo pulp regenerated as fibers is made from bamboo pulp, bamboo fiber, bamboo flock and combinations thereof.

6. The wound site dressing according to claim 1, wherein said wound dressing composition containing bamboo pulp and/or bamboo pulp regenerated as fibers and/or a derivative of said bamboo pulp and/or bamboo pulp regenerated as fibers is made from bamboo pulp, bamboo fiber, bamboo flock and combinations thereof in combination with natural and synthetic polymeric absorbents, hydrocolloid/polysaccharide absorbents, gum absorbents, resin absorbents, inorganic absorbents, hydrogel absorbents and combinations thereof.
